(19) United States
(12) Patent Application Publication
(10) Pub. No.: US 2019/0255075 A1
(43) Pub. Date: Aug. 22, 2019

(54) TREATMENT OF PROSTATE CANCER

(71) Applicant: James Henry Hall, Oconomowoc, WI (US)

(72) Inventor: James Henry Hall, Oconomowoc, WI (US)

(21) Appl. No.: 16/274,664

(22) Filed: Feb. 13, 2019

Related U.S. Application Data
(60) Provisional application No. 62/632,905, filed on Feb. 20, 2018.

(51) Int. Cl. A61K 31/58; A61P 35/00; A61K 31/765; A61K 31/436

(52) U.S. Cl. CPC A61K 31/58; A61K 31/436; A61K 31/765; A61P 35/00

(57) ABSTRACT

Novel combination therapy for treating prostate cancer, and in particular, treatment resistant prostate cancer, is described. The novel combination therapy includes administering a combination of active agents including a 5-alpha reductase inhibitor (e.g., finasteride), and a steroidal 17-alpha spirolactone (e.g., spironolactone). The novel combination therapy may additionally include sirolimus.

### CROSS REFERENCE TO RELATED APPLICATIONS

This application claims the benefit of U.S. Provisional Application No. 62/630,905 filed Feb. 15, 2018, the contents of which are expressly incorporated herein by reference in its entirety.

### TECHNICAL FIELD

The present disclosure relates to the treatment of prostate cancer using a novel combination therapy.

### BACKGROUND

Prostate cancer is cancer that develops in the prostate gland, which is a gland of the male reproductive system. While a majority of prostate cancers are slow growing, some grow relatively quickly and spread from the prostate to other parts of the body, including the bones and the lymph nodes. Although prognosis for localized prostate cancer is good, prostate cancer is difficult to cure once the cancer becomes aggressive and metastasizes.

Men who are diagnosed with low grade forms of prostate cancer, especially those who are elderly, are often followed without treatment, which means that they are under careful observation with the intention of treatment for a cure, if there are signs of cancer progression. Treatment of aggressive cancer involves surgery, radiation therapy, external beam radiation therapy, high intensity focused ultrasound, chemotherapy including oral chemotherapeutic agents, cryosurgery, hormonal therapy, or a combination thereof. Although androgen deprivation therapy (ADT) through either chemical or surgical castration works initially to control metastatic prostate cancer, the cancer often progresses to treatment resistant prostate cancer, such as castration resistant prostate cancer (CRPC) or hormone refractory prostate cancer (HRPC). At the present, there is no efficient therapy available for treatment resistant prostate cancer.

Prostate cancer is the second leading cause of cancer related death of men in the U.S. Accordingly, improved therapies are needed to treat prostate cancer, especially CRPC.

### SUMMARY

The present disclosure provides a novel combination therapy for treating prostate cancer, and in particular, treatment resistant prostate cancer. In embodiments, the therapy includes administering a combination of active agents including a 5-alpha reductase inhibitor (e.g., finasteride), a steroidal 17-alpha spirolactone (e.g., spironolactone), and sirolimus. In other embodiments, the therapy includes administering a combination of a 5-alpha reductase inhibitor (e.g., finasteride) and a steroidal 17-alpha spirolactone (e.g., spironolactone). In embodiments, the novel combination therapy can be used to treat biochemical recurrence of prostate cancer, and/or decrease the PSA level of a subject with prostate cancer.

### BRIEF DESCRIPTION OF THE DRAWINGS

FIGS. 1A and 1B show the subject's PSA levels. FIG. 1A shows a table of the subject's PSA levels measured between October 2008 and February 2018. FIG. 1B shows a chart of the values in FIG. 1A.

### DETAILED DESCRIPTION

The present disclosure describes a novel combination for treating prostate cancer, including treatment resistant prostate cancer. In embodiments, the combination therapy includes at least two active agents, a 5-alpha reductase inhibitor and a steroidal 17-alpha spirolactone. In particular embodiments, the combination therapy includes a third active agent, sirolimus.

The first active agent of the combination therapy is a 5-alpha reductase inhibitor. 5-alpha reductase inhibitors are active agents that block the action of 5-alpha reductase, an enzyme that converts testosterone into dihydrotestosterone (DHT). The use of 5-alpha reductase inhibitors increases the level of testosterone and decreases the level of DHT. An increased amount of DHT has been associated with androgenetic alopecia (male-pattern baldness), benign prostatic hyperplasia (BPH), and prostate cancer. 5-alpha reductase inhibitors have been approved for the treatment of androgenetic alopecia and BPH, but not for the treatment of prostate cancer. Examples of 5-alpha reductase include finasteride and duasteride.

Finasteride was the first 5-alpha reductase inhibitor discovered. Finasteride is a synthetic androstane steroid and 4-azasteroid. It is an analog of androgen steroid hormones, such as testosterone and DHT. Finasteride was approved for the treatment of BPH in 1992 and for the treatment alopecia in 1997. Finasteride is marketed under the brand names PROSCAR® (Merck & Co., Inc., Whitehouse Station, N.J.), PROPECIA® (Merck & Co., Inc.), and others. The chemical structure of finasteride is shown in Formula (I):

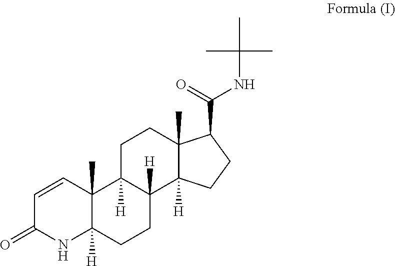

Chemical names for finasteride include 17β-(N-tert-Butylcarbamoyl)-4-aza-5α-androst-1-en-3-one and N-(1,1-Dimethylethyl)-3-oxo-4-aza-5α-androst-1-ene-17β-carboxamide. Other names include MK-906 and L-652,931.

Duasteride is sold under the brand name AVODART® (GLAXOSMITHKLINE LLC, Wilmington, Del.) and others. The chemical structure of duasteride is shown in Formula (II):

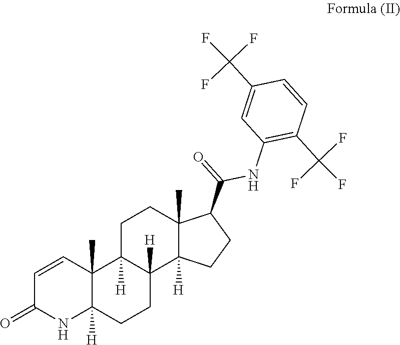

Chemical names for duasteride include N-[2,5-Bis(trifluoromethyl)phenyl]-3-oxo-4-aza-5α-androst-1-ene-17β-carboxamide. Other names include GG-745, GI-198745 and GI-198745X. Duasteride is an analog of finasteride.

Examples of analogs and derivatives of finasteride and duasteride include compounds disclosed in WO 2006058781.

The second active agent of the combination therapy is a steroidal 17-alpha spirolactone. This family of compounds are steroids that are both spiro compounds and lactones. They are antimineralalocorticoids or antagonists of mineralocorticoid receptor and have been used as potassium-sparing diuretics. Potassium-sparing diuretics, as the name implies, are diuretics that do not promote the secretion of potassium into the urine. These drugs are used in combination with other drugs for the treatment of hypertension and management of congestive heart failure. At low doses, these drugs have been found to be effective clinically in reducing blood pressure. Examples of steroidal 17-alpha spirolactone include spironolactone, eplerenone, and canrenone.

Spironolactone was first synthesized in 1957 and was first marketed as an antimineralocorticoid. It is marketed under the brand names ALDACTONE® (PHARMACIA & UPJOHN COMPANY, LLC, North Peapack, N.J.), Spiractin, Verospiron, and others. Other uses for spironolactone include treatment for heart failure, high blood pressure, edema, and acne. In addition, it is a steroid that blocks the effects of hormones aldosterone and testosterone and also has some estrogen-like effects. It has been given as part of a feminizing hormone therapy in transgender women. The chemical structure of spironolactone is shown in Formula (III):

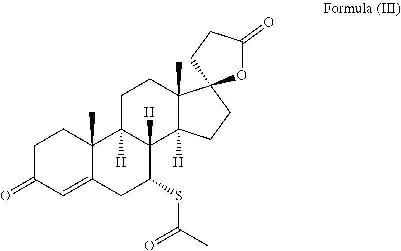

Chemical names for spironolactone include 7α-Acetylthiospirolactone, 7α-Acetylthio-17α-hydroxy-3-oxopregn-4-ene-21-carboxylic acid γ-lactone, 7α-Acetylthio-3-oxo-17α-pregn-4-ene-21,17β-carbolactone, 3-(3-Oxo-7α-acetylthio-17β-hydroxyandrost-4-en-17α-yl)propionic acid lactone, 7α-Acetylthio-17α-(2-carboxyethyl)androst-4-en-17β-ol-3-one γ-lactone, and 7α-Acetylthio-17α-(2-carboxyethyl)testosterone γ-lactone). Other names include SC-9420 and NSC-150339.

Examples of analogs and derivatives of spironolactone include those that are closely related structurally such as canrenone, potassium canrenoate, drospirenone, and eplerenone, which are used clinically. Other examples include never-marketed spirolactones SC-5233 (6,7-dihydrocanrenone; 7α-desthioacetylspironolactone), SC-8109 (19-nor-6,7-dihydrocanrenone), spiroxasone, prorenone (SC-23133), mexrenone (SC-25152, ZK-32055), dicirenone (SC-26304), spirorenone (ZK-35973), and mespirenone (ZK-94679).

In embodiments, the combination therapy includes a third active agent, sirolimus. Sirolimus was first isolated in 1972 from Streptomyces hygroscopicus and developed as an antifungal agent. However, its use as an antifungal agent was abandoned when it was discovered that sirolimus has immunosuppressive and antiproliferative properties due to its ability to inhibit mTOR. Sirolimus has immunosuppressant functions that prevent organ transplant rejection. It inhibits activation of T cells and B cells by reducing the production of interleukin-2 (IL-2). Sirolimus was originally approved for the prevention of organ transplant rejection. In 2015, the FDA approved sirolimus for the treatment of lymphangioleiomyomatosis (LAM), a rare progressive lung disease that affects women of childbearing age. Because of its antiproliferative effect, sirolimus has also been used to coat coronary stents to prevent restenosis in coronary arteries following balloon angioplasty. Sirolimus has also been used as a topical treatment for Kaposi's sarcoma (Saggar, S. et al., Arch Dermatol. 2008 May; 144(5):654-7).

Sirolimus (also known as rapamycin and marketed under the brand name RAPAMUNE® (Wyeth, LLC, New York, N.Y.)), is a macrolide having the chemical structure as shown in Formula (IV):

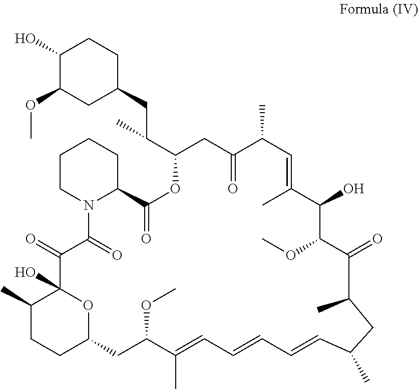 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

Other names for sirolimus include AY 22989 and 12190A. Examples of analogs and derivatives of sirolimus include tacrolimus, 1, 2,3,4-tetrhydro-rapamycin, everolimus, and temsirolimus.

Sirolimus has had disappointing effects when tested as a single agent cancer therapy. Sirolimus was tested for patients with hormone refractory prostate cancer (Amato, R., et al., Clin Genitourin Cancer. 2008 September; 6(2):97-102), and the results showed that, of twelve evaluable patients with hormone refractory prostate cancer, only two patients had positive outcomes. One of the two patients had a decrease in PSA of 50% and a partial radiographic response. The other patient had only a decrease in PSA but not a radiographic response.

Sirolimus has also been tested with cisplatin, a platinum-based chemotherapeutic agent, as a combination therapy to treat prostate cancer cells (Imrali, A, et al., Am J Cancer Res. 2016 Aug. 1; 6(8):1772-84). The studied found that when sirolimus was used alone for the treatment of prostate cancer cells it had limited effects, but the combination of sirolimus and cisplatin was significantly more effective than cisplatin treatment alone. It was hypothesized that the effectiveness of the cisplatin/sirolimus combination was because sirolimus caused the cells to be more sensitive to cell growth-limiting effects of cisplatin, by decreasing cyclin D expression.

In embodiments, the combination therapy includes at least two active agents, finasteride and spironolactone. In particular embodiments, the combination therapy includes at least three active agents: finasteride, spironolactone, and sirolimus

The term “finasteride” includes functional derivatives, functional analogs, as well as pharmaceutically acceptable salts, esters or amides thereof.

The term “spironolactone” includes functional derivatives, functional analogs, as well as pharmaceutically acceptable salts, esters or amides thereof.

The term “sirolimus” includes functional derivatives, functional analogs, as well as pharmaceutically acceptable salts, esters or amides thereof.

The term “analog” (also “structural analog” or “chemical analog”) is used to refer to a compound that is structurally similar to another compound but differs with respect to a certain component, such as an atom, a functional group, or a substructure.

The term “derivative” in chemistry refers to a compound that is obtained from a similar compound or a precursor compound by a chemical reaction.

The term “pharmaceutically acceptable salts” refers to salts of the compounds described herein prepared from pharmaceutically acceptable non-toxic bases or acids, including inorganic or organic bases and inorganic or organic acids. Examples of such inorganic bases include aluminum, ammonium, calcium, copper, ferric, ferrous, lithium, magnesium, potassium, sodium, zinc, and the like, and examples of such organic bases include N,N′-dibenzylethylene-diamine, chloroprocaine, choline, diethanolamine, ethylenediamine, N-methylglucamine, lysine, arginine, procaine, and any pharmaceutically acceptable organic bases. Examples of such inorganic acids include hydrochloric, hydrobromic, hydroiodic, nitric, carbonic, sulfuric and phosphoric acid, and examples of such organic acids can be selected from aliphatic, cycloaliphatic, aromatic, arylaliphatic, heterocyclic, carboxylic and sulfonic classes of organic acids.

The active agents of the combination therapies may be formulated using one or more carriers, excipients (e.g., stabilizers), and/or one or more preservatives. In embodiments, the active agents may be formulated for oral administration.

Methods disclosed herein include treating subjects (humans, veterinary animals (dogs, cats, reptiles, birds, etc.), livestock (horses, cattle, goats, pigs, chickens, etc.), and research animals (monkeys, rats, mice, fish, etc.)). Subjects in need of a treatment (in need thereof) are subjects diagnosed with prostate cancer.

Prostate cancer is one of the most common types of cancer in men. Early prostate cancer is often asymptomatic, but prostate cancer (especially at advanced stages) may present with symptoms such as difficulty urinating, pain during urination, decreased urine flow, and/or blood in the urine (hematuria). Screening for prostate cancer may include a digital rectal exam, during which a doctor may examine the prostate for texture or shape abnormalities. Screening for prostate cancer may also include a prostate specific antigen (PSA) test. Abnormalities found during a digital rectal exam and/or an abnormally high PSA level (>4 ng/mL) may lead to further testing for prostate cancer, such as an ultrasound, a biopsy, and/or an MRI.

Following a prostate cancer diagnosis, the prostate tumor may be evaluated to determine the aggressiveness of the cancer. A Gleason score is a commonly used method to evaluate prostate cancer, and includes the sum of two numbers: a first score associated with the cell type of the majority of cells in the tumor, and a second score associated with a less frequent cell type in the tumor. The scoring system includes scores 1-5, with five associated with the most aggressive tumor cells, and a Gleason score can range between 2 and 10, with a score of 10 associated with the most aggressive tumors. Gleason scores of 6 or lower can be used to identify prostate cancers as prognostic grade I, a Gleason score of 3+4=7 can identify the prostate cancer as prognostic grade II, a Gleason score of 4+3=7 can identify the prostate cancer as prognostic grade III, a Gleason score of 8 can identify the prostate cancer as prognostic grade IV, and Gleason scores of 9 and 10 can identify the prostate cancers as prognostic grade V.

Subjects with prostate cancer of grade I (or a Gleason score of 6 or lower) are often not treated immediately, and instead may be prescribed active surveillance. Active surveillance includes monitoring of the subject's PSA level (e.g., two to three times per year), and may include digital rectal exams and/or biopsies.

Subjects with prostate cancer of grade II or higher are often prescribed an initial treatment immediately. Examples of initial treatments for prostate cancer include radical prostatectomy (prostate removal), radiation therapy (e.g., intensity modulated radiation therapy) alone or in combination with androgen deprivation therapy (ADT), and chemotherapy (alone or in combination with ADT).

ADT can refer to a type of treatment for prostate cancer that suppresses male hormones, called androgens. Prostate cells are dependent on androgens, and therefore, suppression of androgens can reduce or inhibit the growth of prostate cells, such as prostate cancer cells. Examples of ADT include surgical methods and drug-based methods. Surgical ADT involves orchiectomy, which is also known as castration. Surgical ADT can cause the subject's body to no longer produce testosterone, which is needed for the growth of prostate cancer cells. Drug-based ADT can include administration of an LHRH agonist or an LHRH antagonist (e.g., degarelix), a CYP17 inhibitor, and/or an antiandrogen. Antiandrogen refers to a molecule that can block the body's ability to use or respond to androgens. Examples of antiandrogens include cyproterone acetate, flutamide, nilutamide, bicalutamide, and enzalutamide.

Treatment resistant prostate cancer can refer to prostate cancer that is not responsive to, or is no longer responsive to, an initial treatment. Treatment resistant prostate cancer includes castration resistant prostate cancer (CRPC), which is also referred to interchangeably as hormone refractory prostate cancer or androgen-independent prostate cancer. CRPC is prostate cancer that is not responsive to, or is no longer responsive to ADT (surgical and/or drug-based ADT). In embodiments, subjects in need of a treatment include subjects with treatment resistant prostate cancer, which may or may not be metastatic.

The term “PSA level” can refer to the level of PSA measured in a blood sample from a subject. PSA, which is also known as gamma-seminoprotein or kallikrein-3, is an enzyme that is produced and secreted by epithelial cells in the prostate. A small amount of PSA is normally detectable in the bloodstream. However, abnormally high concentrations of PSA in the blood can be associated with prostate cancer. PSA levels are usually reported as nanograms of PSA per milliliter of blood. A PSA level of less than 4 ng/mL in a blood sample is considered within normal range for a subject who has never been diagnosed with prostate cancer. For a subject who has already been diagnosed with prostate cancer, the subject's PSA level may be monitored (e.g., during active surveillance), and increasing PSA levels may indicate prostate cancer progression. During active surveillance, a rising PSA level (e.g., increases in PSA level as measured during three consecutive PSA tests) may lead to initiation of treatment. During or after prostate cancer treatment, a subject's PSA level may be monitored to determine the effectiveness of the treatment.

Following an effective prostate cancer treatment, the PSA level of a subject with prostate cancer may be expected to decrease. For example, the PSA level of a subject may drop to a low level or an undetectable level (e.g., <0.2 ng/mL or <0.1 ng/mL) directly following a treatment (e.g., radiation therapy, a prostatectomy, or ADT), if the treatment was effective. The absolute lowest PSA level of a subject following prostate cancer treatment can be referred to as a PSA nadir. Following an effective prostatectomy, the subject's PSA level is expected to remain very low or undetectable because prostate cells are needed to produce PSA. Effective treatment using a drug-based ADT is expected to cause an initial decrease in a subject's PSA level, and the PSA level may remain at the decreased level for months or years. However, if a subject's PSA level begins to rise following ADT, the rising PSA level may be an indication that the prostate cancer has recurred and/or can indicate CRPC.

Rising PSA levels following or during the course of prostate cancer treatment can be referred to as biochemical recurrence, biochemical failure of treatment, or biochemical relapse. Biochemical recurrence may occur prior to the development of physical symptoms of cancer recurrence. Biochemical recurrence may be defined by the Phoenix method, which defines biochemical recurrence as occurring when the subject's PSA level reaches above the sum of the nadir (lowest PSA level measured following treatment) plus 2 ng/m L.

In embodiments, treatment of a subject with the combination therapy may decrease the subject's prostate specific antigen (PSA) level. In particular embodiments, the combination therapy may decrease the subject's PSA level by more than 40%, more than 50%, more than 60%, or more than 70%. This decrease may be based on a comparison of the PSA level measured after initiation of the combination therapy, as compared to a PSA level measured at the start of the combination therapy or just prior to initiation of the combination therapy (e.g., a PSA level measured no more than three months prior to initiation of the therapy). In particular embodiments, the combination therapy may decrease the subject's PSA level by more than 50% in a time-period of less than six months, less than five months, or less than four months, with the time-period measured, for example, starting from the date that the PSA level was measured at the start of the combination therapy or just prior to initiation of the combination therapy.

Therapeutically effective amounts include those that provide an effective amount. An “effective amount” is the amount of active agent(s) necessary to result in a desired physiological change in vivo or in vitro, such as, for example produces an anti-cancer effect, and/or decreases a subject's PSA level. Therapeutically effective amounts, also referred to herein as doses, for administration can be initially estimated based on results from in vitro assays and/or animal model studies. Such information can be used to accurately determine useful doses in subjects of interest. Particularly useful pre-clinical tests include measure of cell growth, cell death, and/or cell viability.

As used herein, an anti-cancer effect refers to a biological effect, which can be manifested by a decrease in tumor volume, a decrease in the number of cancer cells, a decrease in the number of metastases, an increase in life expectancy, or a decrease of various physiological symptoms associated with the cancerous condition. An anti-cancer effect can be an anti-prostate cancer effect. An anti-cancer effect may be evidenced by a biomarker level, such as a PSA level, that is within a measurement range that is considered normal (e.g., a level that is not associated with cancer). An anti-cancer effect may be evidenced by a rate of change of a biomarker level, such as a PSA level, that is considered a normal rate of change (e.g., a rate that is not associated with cancer, such as no change in the biomarker level). An anti-cancer effect can also be manifested by a decrease in recurrence, such as biochemical recurrence, or an increase in the time before the recurrence.

The combination therapy for treating prostate cancer may include a therapeutically effective amount of finasteride, which may be 20-200 μg/kg. In particular embodiments, the therapeutically effective amount of finasteride may be 5 mg or 10 mg per day. The finasteride may be administered orally.

The combination therapy for treating prostate cancer may include a therapeutically effective amount of spironolactone, which may be 0.2-2 mg/kg. In particular embodiments, the therapeutically effective amount of spironolactone may be 25 mg or 50 mg per day. The spironolactone may be administered orally.

The combination therapy for treating prostate cancer may include a therapeutically effective amount of sirolimus, which may be 10-300 μg/kg. In particular embodiments, the therapeutically effective amount of sirolimus may be 2 mg per day. The sirolimus may be administered orally.

In embodiments, the active agents may be administered separately, and/or may be administered simultaneous or sequentially. For example, each active agent may be separately formulated for oral administration, such as in separate tablets, capsules, or gelcaps, and each active agent may be taken (e.g., swallowed) at the same time of day, or at different times of the day.

The actual dose amount administered to a particular subject can be determined by a physician, based on certain parameters such as physical, physiological and psychological factors including target, body weight, stage of cancer, the type of cancer, previous or concurrent therapeutic interventions, idiopathy of the subject, and route of administration.

The combination therapy or treatment described herein includes monitoring and measuring the subject's PSA level before treatment, during the course of treatment, and/or after treatment. As an example, the subject's PSA level is measured before treatment as a first time-point, and the subject's PSA level is measured again after the beginning of treatment, as a second or later time-point, for comparison. A decrease in PSA level at a later time-point as compared to the first time-point indicates that the combination therapy is effective in decreasing the PSA level of the subject or in treating the subject for prostate cancer, such as biochemical recurrence of prostate cancer, and/or treatment resistant prostate cancer.

Kits. Kits may include one or more containers including one or more active agents including a 5-alpha reductase inhibitor (or analog, derivative, or pharmaceutically acceptable salt thereof) and/or a steroidal 17-alpha spirolactone (or analog, derivative, or pharmaceutically acceptable salt thereof). Kits may also include the active agent sirolimus (or an analog, derivative, or salt thereof). Components of the kits may be in separate containers. The containers may also include instructions for administering the active agents, such as instructions printed on one of the containers, on a sheet, or on a pamphlet.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. In particular embodiments, lack of a material effect is evidenced by lack of a statistically-significant reduction in the embodiment's ability to kill prostate cancer cells in vitro or in vivo.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

The following exemplary embodiments and examples illustrate exemplary methods provided herein. These exemplary embodiments and examples are not intended, nor are they to be construed, as limiting the scope of the disclosure. It will be clear that the methods can be practiced otherwise than as particularly described herein. Numerous modifications and variations are possible in view of the teachings herein and, therefore, are within the scope of the disclosure.

### EXEMPLARY EMBODIMENTS

The following are exemplary embodiments:

1. A method of treating prostate cancer, wherein the method includes administering to a subject in need of treatment a combination of active agents including:

a therapeutically effective amount of 5-alpha reductase inhibitor or an analog, derivative, or a pharmaceutically acceptable salt thereof; and

a therapeutically effective amount of steroidal 17-alpha spirolactone or an analog, derivative, or a pharmaceutically acceptable salt thereof.

2. The method of embodiment 1, wherein the 5-alpha reductase inhibitor is finasteride.
3. The method of embodiment 1 or 2, wherein the steroidal 17-alpha spirolactone is spironolactone.
4. The method of any of embodiments 1-3, wherein the combination of active agents further includes a therapeutically effective amount of sirolimus.
5. The method of any of embodiments 1-4, wherein the active agents are administered separately.
6. The method of any of embodiments 1-5, wherein the active agents are administered simultaneously or sequentially.
7. The method of any of embodiments 1-6, wherein the active agents are administered daily.
8. The method of any of embodiments 1-7, wherein the prostate cancer is treatment resistant prostate cancer.
9. The method of any of embodiments 1-8, wherein the prostate cancer is biochemical recurrence of prostate cancer.
10. A method of decreasing the prostate specific antigen (PSA) level of a subject with prostate cancer, the method including administering to the subject in need thereof a combination of active agents including: a therapeutically effective amount of 5-alpha reductase inhibitor or an analog, derivative, or a pharmaceutically acceptable salt thereof; and a therapeutically effective amount of steroidal 17-alpha spirolactone or an analog, derivative, or a pharmaceutically acceptable salt thereof, thereby decreasing the PSA level of the subject as compared to before administering the combination of active agents.
11. The method of embodiment 10, wherein the combination of active agents further includes a therapeutically effective amount of sirolimus.
12. The method of embodiment 10 or 11, wherein the 5-alpha reductase inhibitor is finasteride.
13. The method of any of embodiments 10-12, wherein the steroidal 17-alpha spirolactone is spironolactone.
14. The method of any of embodiments 10-13, wherein the prostate cancer is treatment resistant prostate cancer.
15. The method of any of embodiments 10-14, wherein decreasing the PSA level includes decreasing the PSA level by more than 40%, more than 50%, more than 60%, or more than 70%.
16. The method of any of embodiments 10-15, wherein decreasing the PSA level includes decreasing the PSA level by more than 50% in less than six months, less than five months, or less than four months.
17. The method of any of embodiments 10-16, wherein the method further includes monitoring the PSA level of the subject.
18. The method of embodiment 17, wherein monitoring includes measuring the PSA level of the subject: (i) before and after administering the combination of active agents and/or (ii) for a period of time.
19. A method of treating biochemical recurrence of prostate cancer in a subject in need thereof, the method including:

administering to a subject diagnosed with biochemical recurrence of prostate cancer a combination of active agents including:

a therapeutically effective amount of 5-alpha reductase inhibitor or an analog, derivative, or a pharmaceutically acceptable salt thereof; and

a therapeutically effective amount of steroidal 17-alpha spirolactone or an analog, derivative, or a pharmaceutically acceptable salt thereof.

20. The method of embodiment 19, wherein the combination of active agents further includes a therapeutically effective amount of sirolimus.
21. The method of embodiment 19 or 20, wherein the 5-alpha reductase inhibitor is finasteride.
22. The method of any of embodiments 19-21, wherein the steroidal 17-alpha spirolactone is spironolactone.
23. The method of any of embodiments 19-22, wherein the method further includes monitoring the subject's PSA level.
24. The method of embodiment 23, wherein monitoring includes measuring the PSA level of the subject: (i) before and after administering the combination of active agents and/or (ii) for a period of time.
25. The method of any of embodiments 19-24, wherein the method further includes detecting biochemical recurrence of the prostate cancer, wherein biochemical recurrence of the prostate cancer occurs when the subject's PSA level at a time-point (a second time-point) is more than 2 ng/mL higher than the subject's PSA level at an earlier time-point (a first time-point).
26. The method of embodiment 25, wherein the subject's PSA level at the earlier time point is a PSA nadir.
27. The method of any of embodiments 19-26, wherein the method further includes monitoring the subject's PSA level after administering the combination, and wherein the subject's PSA level after administering the combination is decreased as compared to the subject's PSA level at the time of biochemical recurrence.
28. A kit for the treatment of prostate cancer, wherein the kit includes a combination of active agents including:

a 5-alpha reductase inhibitor or an analog, derivative, or a pharmaceutically acceptable salt thereof; and

a steroidal 17-alpha spirolactone or an analog, derivative, or a pharmaceutically acceptable salt thereof.

29. The kit of embodiment 28, wherein the kit further includes sirolimus.
30. The kit of embodiment 28 or 29, wherein the 5-alpha reductase inhibitor is finasteride.
31. The kit of any of embodiments 28-30, wherein the steroidal 17-alpha spirolactone is spironolactone.
32. The kit of any of embodiments 28-31, wherein the active agents are packaged in separate containers.
33. The kit of any of embodiments 28-32, wherein the kit further includes instructions for administering the active agents.

### EXAMPLES

Example 1. A 56 year old subject was diagnosed with prostate cancer in 2003. The prostate cancer was diagnosed by biopsy. At the time of diagnosis, the subject had a PSA level of 25 ng/mL and a Gleason score of 7 (3+4).

In December 2004, the subject received three months of intensity-modulated radiation therapy. Following the radiation therapy, the subject's PSA level decreased to a nadir of 0.03 ng/mL and then slowly and steadily increased. The subject's PSA level was monitored two to four times per year beginning in 2008. From 2008 to 2012, the PSA level rose from below 1 ng/mL to above 1.5 ng/mL, as measured Feb. 14, 2012. The steady rise in the subject's PSA levels indicated that the radiation therapy had failed to fully eradicate the prostate cancer.

In March 2012, the subject began a treatment regimen of finasteride (5 mg daily, oral) and spironolactone (25 mg daily, oral). After beginning the finasteride/spironolactone combination therapy, the subject's PSA level sharply decreased, and stayed below 1.5 ng/mL until the Sep. 8, 2014 measurement of 1.62 ng/m L. The steady maintenance of PSA levels below 1.5 ng/mL indicated that the finasteride/spironolactone combination was effective for two and a half years.

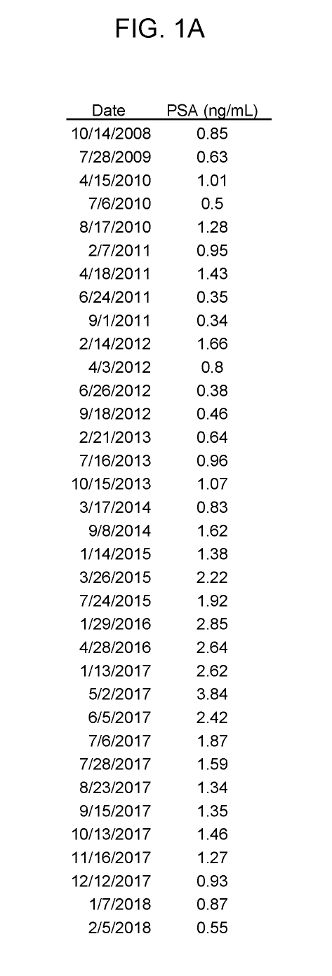

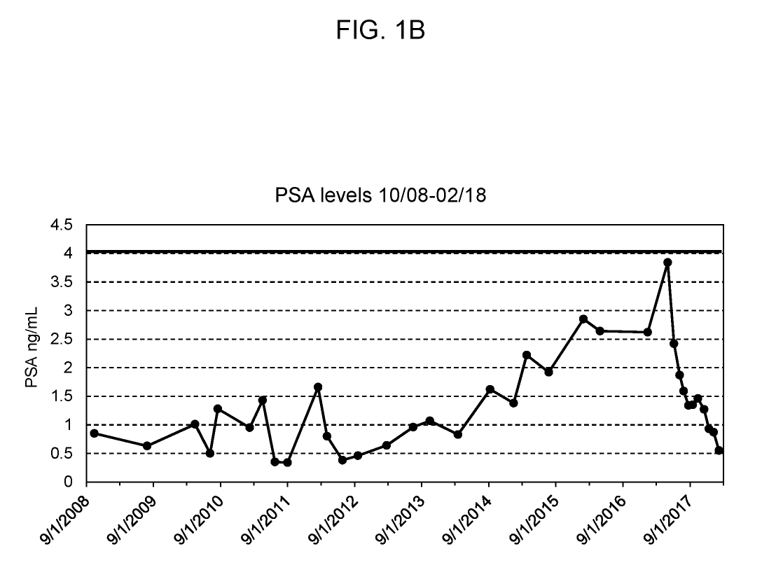

However, the subject's PSA level eventually began to rise again, and on May 2, 2017, the subject's PSA level rose sharply to 3.85 ng/mL (See FIG. 1A). At this point, biochemical recurrence had occurred, as measured by the Phoenix method, and new treatment options were considered.

The subject's treatment regimen was immediately revised to include sirolimus (2 mg daily, oral), in addition to the finasteride/spironolactone combination, and monitoring of the subject's PSA levels was increased to monthly. The next PSA test, taken Jun. 5, 2017, indicated that the subject's PSA level had dropped to 2.42 ng/mL. On Oct. 13, 2017, the dosages of the finasteride and the spironolactone were both doubled (to 10 mg and 50 mg daily, respectively). On Dec. 12, 2017, the subject's testosterone level was measured, with a result of 227.4 ng/dL, which indicated that the subject's treatment regimen effectively suppressed androgen production. The normal testosterone level range for an adult male is 280-1100 ng/dL.

Over the following months, the subject's PSA levels continued to fall to as low as 0.55 ng/mL, as measured on Feb. 5, 2018. FIG. 1A shows a table of the subject's PSA levels measured between October 2008 and February 2018. FIG. 1B shows a chart of the values in FIG. 1A. The drastic decrease in the subject's PSA levels from May 2, 2017 to Feb. 5, 2018 indicates the effectiveness of the finasteride/spironolactone/sirolimus combination therapy for the treatment of prostate cancer.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The subject matter described above is provided by way of illustration only and should not be construed as limiting. Various modifications and changes may be made to the subject matter described herein without following the example embodiments and applications illustrated and described, and without departing from the true spirit and scope of the present invention, which is set forth in the following claims.

All publications, patents and patent applications cited in this specification are incorporated herein by reference in their entireties as if each individual publication, patent or patent application were specifically and individually indicated to be incorporated by reference. While the foregoing has been described in terms of various embodiments, the skilled artisan will appreciate that various modifications, substitutions, omissions, and changes may be made without departing from the spirit thereof.

1. A method of treating prostate cancer, wherein the method comprises administering to a subject in need of treatment a combination of active agents comprising:
a therapeutically effective amount of 5-alpha reductase inhibitor or an analog, derivative, or a pharmaceutically acceptable salt thereof; and
a therapeutically effective amount of steroidal 17-alpha spirolactone or an analog, derivative, or a pharmaceutically acceptable salt thereof.

2. The method of claim 1, wherein the 5-alpha reductase inhibitor is finasteride.

3. The method of claim 1, wherein the steroidal 17-alpha spirolactone is spironolactone.

4. The method of claim 1, wherein the 5-alpha reductase inhibitor is finasteride and the steroidal 17-alpha spirolactone is spironolactone.

5. The method of claim 1, wherein the combination of active agents further comprises a therapeutically effective amount of sirolimus.

6. The method of claim 5, wherein the 5-alpha reductase inhibitor is finasteride and the steroidal 17-alpha spirolactone is spironolactone.

7. The method of claim 1, wherein the active agents are administered separately.

8. The method of claim 7, wherein the active agents are administered simultaneously or sequentially.

9. The method of claim 1, wherein the active agents are administered daily.

10. The method of claim 1, wherein the prostate cancer is treatment resistant prostate cancer.

11. A method of decreasing the prostate specific antigen (PSA) level of a subject with prostate cancer, the method comprising:
administering to the subject in need thereof a combination of active agents comprising:
a therapeutically effective amount of 5-alpha reductase inhibitor or an analog, derivative, or a pharmaceutically acceptable salt thereof, and
a therapeutically effective amount of steroidal 17-alpha spirolactone or an analog, derivative, or a pharmaceutically acceptable salt thereof,
thereby decreasing the PSA level of the subject as compared to before administering the combination of active agents.

12. The method of claim 11, wherein the combination of active agents further comprises a therapeutically effective amount of sirolimus.

13. The method of claim 11, wherein the 5-alpha reductase inhibitor is finasteride.

14. The method of claim 11, wherein the steroidal 17-alpha spirolactone is spironolactone.

15. The method of claim 11, wherein the prostate cancer is treatment resistant prostate cancer.

16. The method of claim 11, wherein decreasing the PSA level of the subject comprises decreasing the PSA level by more than 50%.

17. A kit for the treatment of prostate cancer, wherein the kit comprises a combination of active agents comprising:
a 5-alpha reductase inhibitor or an analog, derivative, or a pharmaceutically acceptable salt thereof; and
a steroidal 17-alpha spirolactone or an analog, derivative, or a pharmaceutically acceptable salt thereof.

18. The kit of claim 17, wherein the kit further comprises sirolimus.

19. The kit of claim 17, wherein the active agents are packaged in separate containers.

20. The kit of claim 17, wherein the kit further comprises instructions for administering the active agents.
